(19) United States
(12) Patent Application Publication
(10) Pub. No.: US 2020/0087237 A1
(43) Pub. Date: Mar. 19, 2020

(54) METHOD FOR PRODUCING PALLADIUM SALT

(71) Applicant: Heraeus Deutschland GmbH & Co. KG, Hanau (DE)

(72) Inventors: Martin STETTNER, Altenstadt (DE); Hermann VON EIFF, Neuberg (DE); Vasco THIEL, Alzenau (DE); Steffen VOSS, Limeshain (DE)

(21) Appl. No.: 16/690,742

(22) Filed: Nov. 21, 2019

Related U.S. Application Data
(62) Division of application No. 15/504,550, filed on Feb. 16, 2017.

(30) Foreign Application Priority Data
Aug. 19, 2014 (EP) 14181352.7

(51) Int. Cl. C07C 53/10; B22F 1/00; B22F 9/16; C22F 1/14

(52) U.S. Cl. CPC C07C 53/10; B22F 1/0088; B22F 1/0003; B22F 9/16; B22F 2301/25; B22F 2999/00; B22F 2998/10; B22F 2201/013; C22F 1/14

(57) ABSTRACT

The present invention relates to a method for the production of palladium(0) powder in which a palladium(0) starting powder is subjected to a thermal treatment in a furnace at a temperature of no more than 370° C. in a hydrogen gas atmosphere.

### CROSS-REFERENCE TO RELATED APPLICATIONS

This application is a Section 371 of International Application No. PCT/EP2015/068294, filed Aug. 18, 2015, which was published in the German language on Feb. 25, 2016 under International Publication No. WO 2016/026847 A1 and the disclosure of which is incorporated herein by reference.

### BACKGROUND OF THE INVENTION

The present invention relates to a method for the production of an active palladium(0) powder.

Powdered palladium is used in many different applications, for example as a catalyst or as an educt for conversion with suitable reaction partners.

Palladium(0) powder (i.e. powdered metallic palladium of oxidation stage 0) and/or palladium(0) sponges are used in applications including the synthesis of palladium salts, such as, e.g., palladium(II) nitrate or palladium(II) carboxylate (e.g. palladium(II) acetate or palladium(II) propionate). During the production of palladium(II) nitrate, for example, palladium(0) powder is converted with nitric acid. During the production of palladium(II) acetate, for example, palladium(0) powder is converted with acetic acid and nitric acid in accordance with the following reaction equation:

3Pd+6HNO₃+6HOAc→Pd₃(OAc)₆+6NO₂+6H₂O

For the process to be efficient, it is desired that the reaction initiates at an already relatively low temperature (e.g. already at room temperature) and that additional external heating of the starting mixture is not required or can at least be minimized. However, for this purpose it is necessary that the palladium(0) powder possesses a sufficiently high activity for this reaction.

It is generally known that palladium(0) powders are accessible through various production methods.

Palladium(0) powder can be produced, for example, by thermal decomposition of diaminedichloro-palladium(II). Palladium(0) powder can also be obtained by reducing hexa- or tetrachloropalladiate with formic acid.

Moreover, it is generally known to reduce halogen-containing palladium compounds to palladium with hydrazine. Accordingly, for example, DE 102 49 521 describes a method for the production of palladium in which a halogen-containing palladium compound is reduced to palladium(0) powder by hydrazine and/or the derivatives thereof, and the palladium(0) powder thus obtained is heated to a temperature of 550-1,200° C. in a nitrogen atmosphere.

However, it has been evident that the palladium(0) powders produced according to the conventional methods are not sufficiently active to initiate the reaction during the production of palladium salts, such as, e.g., palladium(II) nitrate or palladium(II) carboxylates, already at a relatively low temperature (preferably already at room temperature).

### BRIEF SUMMARY OF THE INVENTION

Accordingly, one object of the present invention is to provide a palladium(0) powder that comprises the highest possible activity, in particular for the production of palladium salts.

This object is met by a method for the production of palladium(0) powder in which a palladium(0) starting powder is subjected to a thermal treatment in a furnace at a temperature of no more than 370° C. in a hydrogen gas atmosphere.

### DETAILED DESCRIPTION OF THE INVENTION

As shall be described in more detail below, it has been noted, surprisingly, in the scope of the present invention that, upon treatment of a Pd starting powder in a hydrogen gas atmosphere, relatively low temperatures (370° C. or less) are already sufficient to obtain a very active palladium(0) powder. This palladium(0) powder can be used to initiate the conversion reaction leading to a palladium salt, such as, e.g., palladium(II) nitrate or palladium(II) carboxylate, already at room temperature. Moreover it has been noted, surprisingly, in the scope of the present invention that maintaining said relatively low treatment temperature is essential with regard to the activity and that thermal hydrogen treatment at a temperature above 370° C. leads to a palladium(0) powder of clearly lower activity.

The temperature of the thermal treatment refers to the temperature on the interior of the furnace.

The term “palladium(0)” refers to palladium of oxidation number 0, i.e., metallic palladium.

In the scope of the present invention, the term “palladium(0) powder” shall also comprise a palladium(0) sponge. As is known to a person skilled in the art, a palladium sponge is a relatively coarse-grained form of palladium. In the scope of the present invention, a powder shall be understood to also mean a material in which the powder particles are sintered together at least in part and in which the material, therefore, is particulate, but no more or only partially pourable or flowable.

Methods for the production of a palladium(0) starting powder are known to a person skilled in the art.

The palladium(0) starting powder can be obtained, for example, by reducing a Pd(II) compound or a Pd(IV) compound. Preferably, this concerns a halogen-containing Pd(II) or Pd(IV) compound, such as, e.g., PdCl₂, (NH₄)₂PdCl₆, (NH₄)₂PdCl₄, Pd(NH₃)₄Cl₂, Pd(NH₃)₂Cl₂.

Hydrazine, hydrazinium salts, organic hydrazine derivatives or formic acid can be specified as exemplary reducing agents. The production of palladium through the use of hydrazine as reducing agent is described, for example, in DE 102 49 521 A1.

Palladium(0) starting powder can also be produced by thermal decomposition of diaminedichloro-palladium(II).

As an option, the palladium(0) starting powder can first be dried before subjecting it to the thermal hydrogen treatment in the furnace. This drying step can proceed, for example, in the furnace or, just as well, outside the furnace. As a matter of principle, it is also feasible to subject a still-wet palladium(0) starting powder to the thermal hydrogen treatment.

As mentioned above, the thermal treatment of the palladium(0) starting powder proceeds in a furnace in a hydrogen gas atmosphere.

To implement the thermal treatment, the palladium(0) starting powder is preferably introduced into a furnace and hydrogen gas is allowed to flow into the furnace such that the palladium(0) starting powder is present in a hydrogen gas atmosphere.

In the scope of the present invention, a furnace shall be understood to be a device that comprises a space enclosed by a wall or walls, as the case may be (interior of the furnace), in which heat can be supplied in controlled manner to an object to be subjected to a thermal treatment.

Suitable furnaces for a thermal treatment of this type are generally known to a person skilled in the art and are commercially available. The heating of the furnace can be controlled and checked by appropriate control technology. Preferably, the measuring elements for temperature determination are attached appropriately such that the temperature in the interior of the furnace can be determined reliably and such that the risk of overheating can thus be reduced. A tube furnace shall be mentioned here as an exemplary furnace. Other types of furnaces are just as suitable, though.

On principle, the hydrogen content of the hydrogen gas atmosphere can vary across a broad range. At a lower hydrogen content, a longer duration of treatment of the palladium(0) starting powder in the hydrogen gas atmosphere may need to be selected in order for the palladium(0) powder to have sufficient activity (e.g., for later conversion with a mineral acid). The hydrogen content of the hydrogen gas atmosphere (i.e., the hydrogen content of the interior of the furnace) is, for example, at least 5% by volume, more preferably at least 10% by volume or at least 20% by volume, even more preferably at least 30% by volume or at least 50% by volume, yet more preferably at least 70% by volume or even at least 90% by volume, relative to the total amount of gases present in the hydrogen gas atmosphere. Provided other gases are present, these may be, for example, inert auxiliary gases (e.g., N2) or inevitable residual amounts of air. The content of oxygen (e.g., of air that is still present) is preferably kept as low as possible, e.g., less than 1% by volume, more preferably less than 0.1% by volume or even less than 0.01% by volume, relative to the total amount of gases present in the hydrogen gas atmosphere.

The flow of hydrogen into the furnace can proceed continuously or, alternatively, discontinuously. Preferably, a continuous hydrogen flow in the furnace is evident during the entire thermal treatment of the palladium(0) starting powder. As shall be illustrated later on, though, it may be preferred to stop the hydrogen supply to the furnace after completion of the thermal treatment during the cooling phase of the furnace, and to supply, instead, an inert gas, such as, e.g., nitrogen, into the furnace during the cooling phase.

Once a hydrogen gas atmosphere is established in the furnace, the furnace temperature is increased, although a temperature of 370° C. must not be exceeded.

As has already been mentioned above, the activity of the palladium(0) powder is clearly reduced in the production procedure of palladium salts if the hydrogen treatment of the palladium(0) starting powder is performed above 370° C.

Suitable measures preventing overheating of a furnace are generally known to a person skilled in the art. The furnace can be prevented from heating up, for example, by running one or more temperature ramps. Running a temperature ramp involves heating the furnace to a holding temperature T₁ and maintaining the holding temperature T₁, as constant as possible for a period of time t₁. If a second temperature ramp is run as well, there is another heating process from the first holding temperature T₁ to a second holding temperature T₂ and this holding temperature T₂ is subsequently kept as constant as possible for a period of time t₂. Running these temperature ramps provides for approaching the given maximal temperature of the furnace in an appropriate manner such that the risk of overheating is minimized. The number of temperature ramps, suitable holding temperatures T₁, T₂, etc., and suitable holding times t₁, t₂, etc., can be selected appropriately and readily by a person skilled in the art such that overheating of the furnace to temperatures above 380° C. is prevented. For example 3-10 or 4-8 temperature ramps can be run while heating the furnace to the maximal temperature, whereby the holding temperatures T₁, T₂, etc., can differ by 10-100° C. from each other and the holding times t₁, t₂, etc., can be 5-90 minutes or 15-80 minutes.

Alternatively or in addition to the use of temperature ramps, the risk of overheating can also be minimized by low heating rates.

Preferably, the thermal hydrogen gas treatment in the furnace proceeds at a temperature of no more than 360° C., more preferably no more than 350° C.

The preferred lower temperature limit for the thermal hydrogen gas treatment in the furnace is 150° C., more preferably 230° C., yet more preferably 280° C.

Accordingly, the thermal treatment of the palladium(0) starting powder preferably proceeds at a temperature in the range of 150° C. to 370° C., more preferably in the range of 230° C. to 360° C. or of 280° C. to 350° C.

The duration of the thermal treatment of the palladium(0) starting powder in the hydrogen gas atmosphere can vary over a broad range and also depends on the amount of palladium(0) starting powder that is used. The duration of the thermal treatment should be selected appropriately such that the palladium(0) powder thus obtained possesses sufficiently high activity in a production method for palladium salts. Since the activity of a palladium(0) powder for the production of a palladium salt can be tested readily (for example, by a small-scale test reaction or by thermo-gravimetric analysis of the Pd(0) powder), the optimal period of time for the thermal treatment of the Pd(0) starting powder can be determined readily.

After the thermal treatment, the furnace is allowed to cool down (e.g., to room temperature) and the palladium(0) powder can subsequently be removed and used for the production of palladium salts. Preferably, during the cooling process, no further amounts of hydrogen, but rather an inert gas, such as, e.g., nitrogen or a noble gas, is supplied into the furnace.

After the furnace has cooled down, the palladium(0) powder can, in addition, be subjected to a post-treatment, for example to a mechanical disintegration and/or a grinding process.

If the palladium(0) powder is not used immediately for the production of the palladium salt, it can be advantageous to store the palladium(0) powder in an inert gas atmosphere (e.g., a N2 atmosphere).

In a further aspect, the present invention relates to a palladium(0) powder that is and/or can be obtained according to the method described above.

As shall be described in more detail below, the palladium(0) powder produced in the method according to the invention does not only show improved activity in the production of palladium salts, but also a characteristic mass increase when heated and/or annealed while exposed to air, which makes it different from other palladium(0) powders.

In a further aspect, the present invention therefore relates to a palladium (0) powder that exhibits an increase in mass of at least 13.0% by weight when heated up to a temperature of 990° C. while being exposed to air.

The increase in mass can be determined by thermo-gravimetry. The heating rate is, e.g., 10° C./min. The palladium(0) powder is heated from a starting temperature, which is usually 25° C., up to a temperature of 990° C. and the increase in mass proceeding in this temperature interval is determined. The thermo-gravimetric measurement is done in an air atmosphere.

Preferably, the increase in mass is at least 13.5% by weight, more preferably at least 14.0% by weight.

Using this characteristic increase in mass of the palladium(0) powder according to the invention allows a thermo-gravimetric analysis to be used to determine very rapidly whether a certain palladium(0) powder shows sufficiently high activity for the production of palladium salts.

In a further aspect, the present invention relates to the use of the palladium(0) powder described above as an educt for the production of a palladium salt.

The palladium salt can be either a palladium(II) salt or a palladium(IV) salt.

Exemplary salts are palladium salts of mineral acids (e.g. Pd(II) nitrate, Pd(II) sulfate or Pd(II) chloride) and palladium(II) salts of carboxylic acids (e.g. C₂₋₈ carboxylic acids) such as, e.g., palladium acetate or palladium propionate.

In a further aspect, the present invention relates to a method for producing a palladium salt, comprising
(i) providing a palladium(0) powder according to the method described above, and
(ii) reacting the palladium(0) powder with a mineral acid.

As mentioned above, the palladium salt can be either a palladium(II) salt or a palladium(IV) salt. With regard to exemplary salts, reference is made to the explanations made above.

Step (i) involves providing a palladium(0) powder according to the method described above.

Step (ii) involves converting the palladium(0) powder with a mineral acid. Suitable reaction conditions for the conversion of the palladium(0) powder with a mineral acid are known to a person skilled in the art. Suitable mineral acids include, for example, nitric acid, sulfuric acid, hydrochloric acid or a mixture of at least two of these mineral acids (e.g., a mixture of nitric acid and hydrochloric acid). Nitric acid is a preferred mineral acid.

A preferred embodiment is a method for the production of palladium(II) nitrate, whereby the palladium(0) powder is converted with nitric acid in step (ii). The palladium(II) nitrate can be used for further conversions, for example for the production of further salts, by replacing the nitrate by another anion.

It is feasible just as well in the scope of the present invention to have at least one further reaction partner be present in step (ii) in addition to the mineral acid (e.g., nitric acid). In a preferred embodiment, the conversion in step (ii) proceeds in the presence of a carboxylic acid or of a carboxylic acid anhydride or of a mixture thereof. A palladium(II) carboxylate can be produced by this means.

Preferably, the palladium(II) carboxylate is a palladium(II) C₂₋₈ carboxylate, such as, e.g., palladium acetate or palladium propionate. Accordingly, the carboxylic acid is preferably a C₂₋₈ carboxylic acid, such as, e.g., acetic acid or propionic acid. Anhydrides of these carboxylic acids can be present in step (ii) just as well.

In a preferred embodiment, the mineral acid is nitric acid and the carboxylic acid is acetic acid. Due to the presence of these reaction partners in step (ii), palladium(II) acetate is obtained.

Suitable reaction conditions for the conversion of the palladium(0) powder with a mineral acid (such as, e.g., nitric acid) and a carboxylic acid (such as, e.g., acetic acid) are known to a person skilled in the art.

If the palladium(0) powder according to the invention is used, the reaction is initiated already at a relatively low temperature, e.g. is initiated already at room temperature.

If applicable, the starting mixture containing the educts can be heated somewhat in order to start the reaction.

The invention shall be illustrated in more detail based on the following examples.

### EXAMPLES

The same palladium(0) starting powder was used in all experiments below and was produced as follows in accordance with the example of DE 102 49 521 A1:

Pd(NH₃)₂Cl₂ was transferred to a beaker and hot, deionized water was added until the suspension was easy to stir. Subsequently, 5-10 mL ammonia solution (25% solution) were added while stirring such that a slightly alkaline solution was generated. Then, 30-60 mL hydrazine solution (22% solution) were added slowly and in aliquots. The suspension foams during the addition of hydrazine. The addition of hydrazine must be adapted to the foaming. Another 3 mL of hydrazine solution were added as an excess. Subsequently, stirring was continued for one more hour and the Pd sponge thus generated was then filtered through a funnel filter. The Pd sponge was washed approx. 10 times with hot, deionized water. The Pd sponge, still slightly wet, was transferred to quartz glass boats and these were pushed into a lockable tube furnace. The furnace was equipped with an interior tube made of quartz glass. Subsequently, nitrogen gas was supplied through the interior tube. The exit of the tube was connected to a gas washing bottle filled with 2N sulfuric acid. After a period of 10 minutes, in which the oxygen was completely displaced from the interior tube, the oven was heated linearly to a temperature of 250° C. over the course of two hours. Said temperature was maintained for 4 hours and then the furnace was heatedly further linearly to a temperature of 600-650° C. After a holding time of 5 hours, the furnace was allowed to cool down to approx. 50° C. while rinsing with nitrogen. The Pd sponge was removed and mechanically disintegrated.

### Reference Example 1

The palladium(0) starting powder produced according to the method described above was tested for its activity in the production of palladium acetate. The procedure was as follows:

30 mL acetic acid anhydride and 300 mL acetic acid were added to 30 g of the palladium(0) starting powder. Then, nitric acid was added.

There was no formation of NOₓ at room temperature and the palladium(0) powder did not react with acetic acid and nitric acid to form palladium acetate. Even heating to 60° C. did not start the reaction.

### Example 1

The palladium(0) starting powder was placed in a tube furnace. Hydrogen was allowed to flow into the furnace. The flow of H₂ was 2 m³/h. After formation of the hydrogen gas atmosphere, the furnace was heated to a maximal temperature of 340° C. according to the following temperature program:
heating to 100° C.;
holding the temperature of 100° C. for 60 minutes (first temperature ramp);
further heating to 150° C.;
holding the temperature of 150° C. for 30 minutes (second temperature ramp);
further heating to 200° C.;
holding the temperature of 200° C. for 30 minutes (third temperature ramp);
further heating to 280° C.;
holding the temperature of 280° C. for 30 minutes (fourth temperature ramp);
further heating to 300° C.;
holding the temperature of 300° C. for 30 minutes (fifth temperature ramp);
further heating to 340° C. and continuation of the thermal treatment for another 150 minutes;
allowing the furnace to cool down to room temperature.

During the cooling phase, the flow of H₂ was stopped and nitrogen was supplied into the furnace instead.

A part of the palladium(0) powder thus obtained was subjected to a thermo-gravimetric analysis (TG unit: Netzsch TG 209). The heating rate was 10° C./min and the sample was heated in an air atmosphere up to a temperature of 990° C. The sample showed an increase in mass of 14.2% by weight.

A second sample of the palladium(0) powder was taken and again subjected to a thermo-gravimetric analysis under identical conditions. The sample showed an increase in mass of 14.1% by weight.

The remaining palladium(0) starting powder was tested for its activity in the production of palladium acetate. The procedure of Reference example 1 was adopted for this purpose, i.e., 30 mL acetic acid anhydride and 300 mL acetic acid were added to 30 g of the palladium(0) starting powder. Then, nitric acid was added.

There was some formation of NOₓ even without external heating and the palladium(0) powder reacted with acetic acid and nitric acid to form palladium acetate. This demonstrates that the palladium(0) powder according to the invention has very high activity.

### Reference Example 2

The palladium(0) starting powder was placed in a tube furnace. Hydrogen was allowed to flow into the furnace. The flow of H₂ was 2 m³/h. After formation of the hydrogen gas atmosphere, the furnace was heated to a maximal temperature of 380° C. according to the following temperature program:
heating to 100° C.;
holding the temperature of 100° C. for 60 minutes (first temperature ramp);
further heating to 150° C.;
holding the temperature of 150° C. for 30 minutes (second temperature ramp);
further heating to 200° C.;
holding the temperature of 200° C. for 30 minutes (third temperature ramp);
further heating to 280° C.;
holding the temperature of 280° C. for 30 minutes (fourth temperature ramp);
further heating to 300° C.;
holding the temperature of 300° C. for 30 minutes (fifth temperature ramp);
further heating to 380° C. and continuation of the thermal treatment for another 150 minutes;
allowing the furnace to cool down to room temperature.

During the cooling phase, the flow of H₂ was stopped and nitrogen was supplied into the furnace instead.

A part of the palladium(0) powder thus obtained was subjected to a thermo-gravimetric analysis (TG unit: Netzsch TG 209). The heating rate was 10° C./min and the sample was heated in an air atmosphere up to a temperature of 990° C. The sample showed an increase in mass of 11.9% by weight.

The remaining palladium(0) starting powder was tested for its activity in the production of palladium acetate. The procedure of Reference example 1 and example 1 was adopted for this purpose, i.e., 30 mL acetic acid anhydride and 300 mL acetic acid were added to 30 g of the palladium(0) starting powder. Then, nitric acid was added.

There was formation of NOx only with additional external heating to the approx. 80° C. and the palladium(0) powder reacted with acetic acid and nitric acid to form palladium acetate.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims

1.-8. (canceled)

9. A method of producing a palladium salt comprising using a palladium(0) powder as an educt for the production, wherein the palladium(0) powder is obtained by subjecting a palladium(0) starting powder to a thermal treatment in a furnace at a temperature of no more than 370° C. in a hydrogen gas atmosphere.

10. A method for producing a palladium salt, comprising:
(i) producing a palladium(0) powder by subjecting a palladium(0) starting powder to a thermal treatment in a furnace at a temperature of no more than 370° C. in a hydrogen gas atmosphere; and
(ii) converting the palladium(0) powder with a mineral acid.

11. The method according to claim 10, wherein the mineral acid is nitric acid, sulfuric acid, hydrochloric acid or a mixture of at least two of these mineral acids.

12. The method according to claim 10, wherein the conversion in step (ii) proceeds in the presence of a carboxylic acid, a carboxylic acid anhydride, or a mixture thereof.
